(19) United States
(12) Patent Application Publication
(10) Pub. No.: US 2021/0207077 A1
(43) Pub. Date: Jul. 8, 2021

(54) COMPOSITION FOR PROMOTING THE GROWTH OF LACTIC ACID BACTERIA COMPRISING GROWTH FACTORS

(71) Applicant: CELL BIOTECH CO., LTD., Gyeonggi-do (KP)

(72) Inventor: Myung Jun Chung, Seoul (KP)

(21) Appl. No.: 17/108,558

(22) Filed: Dec. 1, 2020

(30) Foreign Application Priority Data
Jan. 2, 2020 (KP) 10-2020-0000255

(51) Int. Cl. C12N 1/20

(52) U.S. Cl. CPC C12N 1/20; C12N 2500/74; C12N 2500/05; C12N 2500/34

(57) ABSTRACT

The present disclosure relates to a composition for promoting the growth of lactic acid bacteria comprising growth factors, and more particularly, to a composition for promoting the growth of lactic acid bacteria comprising growth factors, the composition being capable of efficiently promoting the growth of various lactic acid bacteria strains.

### CROSS-REFERENCE TO RELATED APPLICATION(S)

This application claims priority to Korean Patent Application No. 10-2020-0000255, filed on Jan. 2, 2020, the disclosure of which is incorporated herein by reference in its entirety.

### BACKGROUND

### Technical Field

The present disclosure relates to a composition for promoting the growth of lactic acid bacteria comprising growth factors, and more particularly, to the composition being capable of efficiently promoting the growth of various lactic acid bacteria strains.

### Description of the Related Art

Due to increased demand for probiotics, probiotic lactic acid bacteria have become the second largest market after red ginseng among health functional foods in the health functional food market, and along with this, interest in prebiotics, which promote the growth of lactic acid bacteria, has also increased. However, currently commercially available prebiotics are limited to fructooligosaccharides, and the effects thereof have not been scientifically proven and are also uncertain. Accordingly, the development of scientifically proven compositions for promoting the growth of lactic acid bacteria is necessarily required, and the effects thereof also need to be reliably identified.

Although components or substances that promote the growth of each type of lactic acid bacteria are known, studies on substances or compositions that can be applied universally to various strains are comprehensive studies that require a long time, much effort, a large cost and a lot of manpower, and a study environment, facilities and systems, which satisfy these requirements, are required for these studies.

The growth curve of microorganisms can be largely divided into the following four sequential phases: a lag phase in which microorganisms adapt to the environment and express enzymes necessary for cell division; an exponential phase in which cell division is promoted and microorganisms proliferate; a stationary phase in which the cell number remains constant without further increase; and a death phase in which the cell number decreases due to cell death.

It could be confirmed that a composition for promoting the growth of lactic acid bacteria according to the present disclosure could be applied to various lactic acid bacteria and exhibited a clear effect on stable maintenance of the number of lactic acid bacteria by reducing the duration of the lag phase, increasing the number of lactic acid bacteria in the exponential phase, and increasing the duration of the stationary phase. This effect means that the composition according to the present disclosure is of great help in allowing ingested probiotics to effectively settle in gut and to maintain their function during growth and retention in the gut. In addition, this effect can explain that the composition of the present disclosure comprises a proven growth factor that promotes the growth of lactic acid bacteria, unlike conventional prebiotics composed only of sugars. It is expected that an optimal mixture composition comprising a carbon source and a nitrogen source according to the present disclosure can be used universally as a composition for promoting the growth of various lactic acid bacteria.

### SUMMARY

The present inventors have conducted studies and made efforts to develop a composition capable of efficiently promoting the growth of various lactic acid bacteria strains. As a result, the present inventors have developed a composition for promoting the growth of lactic acid bacteria comprising growth factors, which is capable of promoting the growth of various lactic acid bacteria strains, and have experimentally proved and confirmed that the composition efficiently promotes the growth of lactic acid bacteria, thereby completing the present disclosure.

Therefore, an object of the present disclosure is to provide a composition for promoting the growth of lactic acid bacteria, the composition comprising, as growth factors: a carbon source comprising glucose and fructooligosaccharide; and a nitrogen source comprising yeast extract and isolated soy protein.

Other objects and advantages of the present disclosure will be illustrated in more detail by the following detailed description, the appended claims and the accompanying drawings.

According to one aspect of the present disclosure, the present disclosure provides a composition for promoting the growth of lactic acid bacteria, the composition comprising: a carbon source comprising glucose and fructooligosaccharide; and a nitrogen source comprising yeast extract and isolated soy protein.

As used herein, the term “growth factors” refers to compounds, including the above-described carbon source and nitrogen source, which act as substrates for lactic acid bacteria of the present disclosure and promote the growth of the lactic acid bacteria.

In one embodiment of the present disclosure, the glucose may be comprised in an amount of 20 to 40 wt %, preferably 40 wt %, based on 100 wt % of the composition for promoting the growth of lactic acid bacteria. In addition, the fructooligosaccharide may be comprised in an amount of 20 to 40 wt %, preferably 40 wt %, based on 100 wt % of the composition for promoting the growth of lactic acid bacteria. Furthermore, the yeast extract may be comprised in an amount of 10 to 30 wt %, preferably 15 wt %, based on 100 wt % of the composition for promoting the growth of lactic acid bacteria. Additionally, the isolated soy protein may be comprised in an amount of 5 to 30 wt %, preferably 5 wt %, based on 100 wt % of the composition for promoting the growth of lactic acid bacteria.

In one embodiment of the present disclosure, the weight ratio between the glucose and the fructooligosaccharide in the carbon source may be 1:3 to 3:1, preferably 1:1.

In one embodiment of the present disclosure, the weight ratio between the yeast extract and the isolated soy protein in the nitrogen source may be 1:3 to 3:1, preferably 3:1.

The lactic acid bacteria of the present disclosure may be probiotic lactic acid bacteria in the sense that when they are administered in vivo, they settle in the gut and have a beneficial synergistic effect with the gut microbiota.

In one embodiment of the present disclosure, the lactic acid bacteria that are used in the present disclosure may be one or more lactic acid bacteria selected from the group consisting of lactic acid bacteria of the genus Lactobacillus, lactic acid bacteria of the genus Bifidobacterium, lactic acid bacteria of the genus Streptococcus, and lactic acid bacteria of the genus Pediococcus.

In another embodiment of the present disclosure, the lactic acid bacteria of the genus Lactobacillus may be one or more lactic acid bacteria selected from the group consisting of Lactobacillus casei, Lactobacillus plantarum and Lactobacillus rhamnosus. The Lactobacillus casei is preferably Lactobacillus casei CBT-LC5, more preferably Lactobacillus casei CBT-LC5 (accession number: KCTC 12398BP). The Lactobacillus plantarum is preferably Lactobacillus plantarum CBT-LP3, more preferably Lactobacillus plantarum CBT-LP3 (accession number: KCTC 10782BP). The Lactobacillus rhamnosus is preferably Lactobacillus rhamnosus CBT-LR5, more preferably Lactobacillus rhamnosus CBT-LR5 (accession number: KCTC 12202BP). However, the present disclosure is not limited thereto.

In still another embodiment of the present disclosure, the lactic acid bacteria of the genus Bifidobacterium may be one or more lactic acid bacteria selected from the group consisting of Bifidobacterium longum, Bifidobacterium lactis and Bifidobacterium bifidum. The Bifidobacterium longum is preferably Bifidobacterium longum CBT-BG7, more preferably Bifidobacterium longum CBT-BG7 (accession number: KCTC 12200BP). The Bifidobacterium lactis is preferably Bifidobacterium lactis CBT-BL3, more preferably Bifidobacterium lactis CBT-BL3 (accession number: KCTC 11904BP). The Bifidobacterium bifidum is preferably Bifidobacterium bifidum CBT-BF3, more preferably Bifidobacterium bifidum CBT-BF3 (accession number: KCTC 12199BP). However, the present disclosure is not limited thereto.

In yet another embodiment of the present disclosure, the lactic acid bacteria of the genus Streptococcus may be Streptococcus thermophilus. The Streptococcus thermophilus is preferably Streptococcus thermophilus CBT-ST3, more preferably Streptococcus thermophilus CBT-ST3 (accession number: KCTC 11870BP). However, the present disclosure is not limited thereto.

In still yet another embodiment of the present disclosure, the lactic acid bacteria of the genus Pediococcus may be Pediococcus pentosaceus. The Pediococcus pentosaceus is preferably Pediococcus pentosaceus CBT-SL4, more preferably Pediococcus pentosaceus CBT-SL4 (accession number: 10297BP), but the present disclosure is not limited thereto.

### BRIEF DESCRIPTION OF THE DRAWINGS

FIG. 1 shows the results of evaluating the effect of treatment with a composition for promoting the growth of lactic acid bacteria comprising growth factors on the growth of Lactobacillus casei CBT LC5.

FIG. 2 shows the results of evaluating the effect of treatment with the composition for promoting the growth of lactic acid bacteria comprising growth factors on the growth of Bifidobacterium longum CBT BG7.

FIG. 3 shows the results of evaluating the effect of treatment with the composition for promoting the growth of lactic acid bacteria comprising growth factors on the growth of Bifidobacterium bifidum CBT-BF3.

FIG. 4 shows the results of evaluating the effect of treatment with the composition for promoting the growth of lactic acid bacteria comprising growth factors on the growth of Streptococcus thermophilus CBT-ST3.

### DESCRIPTION OF SPECIFIC EMBODIMENTS

The specific embodiments described herein are representative of preferred embodiments or examples of the present disclosure, and thus the scope of the present disclosure is not limited thereto. It will be apparent to those skilled in the art that modifications and other uses of the present disclosure do not depart from the scope of the present disclosure as defined in the appended claims.

### EXAMPLES

### Experimental Methods

### Example: Selection of Optimal Composition

To promote the growth of non-specific various types of lactic acid bacteria, an optimal mixture composition was determined by culturing each of the following strains using each component, ratio and composition and measuring the viable cell count of each of the strains: Lactobacillus casei CBT-LC5 (accession number: KCTC 12398BP), Lactobacillus plantarum CBT-LP3 (accession number: KCTC 10782BP), Lactobacillus rhamnosus CBT-LR5 (accession number: KCTC 12202BP), Bifidobacterium longum CBT-BG7 (accession number: KCTC 12200BP), Bifidobacterium lactis CBT-BL3 (accession number: KCTC 11904BP), Bifidobacterium bifidum CBT-BF3 (accession number: KCTC 12199BP), Streptococcus thermophilus CBT-ST3 (accession number: KCTC 11870BP), and Pediococcus pentosaceus CBT-SL4 (accession number: KCTC 10297BP). Starter cultures were prepared using BL broth.

### Experimental Example 1: Experiment of Selection of Carbon Source

As shown in Table 1 below, 2 parts by weight of each carbon source was added to 100 parts by weight of BL broth, thereby preparing media.

TABLE 1

| | Control | | | Refined | | Fructo- |
|---|---|---|---|---|---|---|
| Strain name | (BL broth) | Glucose | Fructose | sugar | Lactose | oligosaccharid |
| Lactobacillus casei CBT LC5 | 0% | 2% | 2% | 2% | 2% | 2% |
| Lactobacillus plantarum CBT LP3 | 0% | 2% | 2% | 2% | 2% | 2% |
| Lactobacillus rhamnosus CBT LR5 | 0% | 2% | 2% | 2% | 2% | 2% |
| Bifidobacterium longum CBT BG7 | 0% | 2% | 2% | 2% | 2% | 2% |
| Bifidobacterium lactis CBT BL3 | 0% | 2% | 2% | 2% | 2% | 2% |
| Bifidobacterium bifidum CBT BF3 | 0% | 2% | 2% | 2% | 2% | 2% |
| Streptococcus thermophilus CBT ST3 | 0% | 2% | 2% | 2% | 2% | 2% |
| Pediococcus pentosaceus CBT SL4 | 0% | 2% | 2% | 2% | 2% | 2% |

0.1 to 1 part by weight of each of the starter cultures was inoculated into 100 parts by weight of each prepared medium, and then cultured for 20 hours while the pH was maintained at about 5.0 to 6.5. Next, the viable cell count of each culture was measured, and the results of the measurement are summarized in Table 2 below. As a result, it was confirmed that culture with glucose or fructooligosaccharide exhibited an excellent effect on the growth of the lactic acid bacteria. Thus, glucose or fructooligosaccharide was selected as a carbon source for a subsequent experiment (Table 2).

TABLE 2

| | Control | | | Refined | | Fructo- |
|---|---|---|---|---|---|---|
| Strain name | (BL broth) | Glucose | Fructose | sugar | Lactose | oligosaccharide |
| Lactobacillus casei CBT LC5 | 348 | 650 | 408 | 482 | 405 | 615 |
| Lactobacillus plantarum CBT LP3 | 318 | 613 | 452 | 427 | 550 | 616 |
| Lactobacillus rhamnosus CBT LR5 | 212 | 615 | 486 | 425 | 405 | 580 |
| Bifidobacterium longum CBT BG7 | 363 | 612 | 405 | 464 | 456 | 570 |
| Bifidobacterium lactis CBT BL3 | 410 | 770 | 522 | 472 | 420 | 715 |
| Bifidobacterium bifidum CBT BF3 | 306 | 640 | 434 | 325 | 460 | 660 |
| Streptococcus thermophilus CBT ST3 | 280 | 620 | 402 | 504 | 450 | 640 |
| Pediococcus pentosaceus CBT SL4 | 322 | 650 | 550 | 580 | 520 | 702 |

(unit: ×10⁷ cfu/ml)

### Experimental Example 2: Experiment for Combination of Carbon Sources

As shown in Table 3 below, glucose and fructooligosaccharide were mixed together at the indicated ratios, and then 2 parts by weight of each mixture was added to 100 parts by weight of BL broth, thereby preparing media.

TABLE 3

| | Control | 1:1 | 1:2 | 1:3 | 2:1 | 3:1 |
|---|---|---|---|---|---|---|
| Strain name | (BL broth) | 50%:50% | 33%:67% | 25%:75% | 67%:33% | 75%:25% |
| Lactobacillus casei CBT LC5 | 0% | 2% | 2% | 2% | 2% | 2% |
| Lactobacillus plantarum CBT LP3 | 0% | 2% | 2% | 2% | 2% | 2% |
| Lactobacillus rhamnosus CBT LR5 | 0% | 2% | 2% | 2% | 2% | 2% |
| Bifidobacterium longum CBT BG7 | 0% | 2% | 2% | 2% | 2% | 2% |
| Bifidobacterium lactis CBT BL3 | 0% | 2% | 2% | 2% | 2% | 2% |
| Bifidobacterium bifidum CBT BF3 | 0% | 2% | 2% | 2% | 2% | 2% |
| Streptococcus thermophilus CBT ST3 | 0% | 2% | 2% | 2% | 2% | 2% |
| Pediococcus pentosaceus CBT SL4 | 0% | 2% | 2% | 2% | 2% | 2% |

0.1 to 1 part by weight of each starter culture was inoculated into 100 parts by weight of each prepared medium, and then cultured for 20 hours while the pH was maintained at about 5.0 to 6.5. Next, the viable cell count of each culture was measured, and the results of the measurement are summarized in Table 4 below. As a result, it was confirmed that culture with glucose and fructooligosaccharide at the same mixing ratio (50%: 50%) exhibited a better effect on the growth of the lactic acid bacteria than culture at the other mixing ratios. Thus, the mixing ratio of 50:50 was selected as a mixing ratio for a subsequent experiment (Table 4).

TABLE 4

| | Control | 1:1 | 1:2 | 1:3 | 2:1 | 3:1 |
|---|---|---|---|---|---|---|
| Strain name | (BL broth) | 50%:50% | 33%:67% | 25%:75% | 67%:33% | 75%:25% |
| Lactobacillus casei CBT LC5 | 356 | 725 | 616 | 608 | 620 | 618 |
| Lactobacillus plantarum CBT LP3 | 326 | 726 | 612 | 610 | 625 | 660 |
| Lactobacillus rhamnosus CBT LR5 | 255 | 735 | 621 | 622 | 621 | 612 |
| Bifidobacterium longum CBT BG7 | 330 | 722 | 650 | 680 | 640 | 650 |
| Bifidobacterium lactis CBT BL3 | 436 | 729 | 610 | 615 | 610 | 620 |
| Bifidobacterium bifidum CBT BF3 | 302 | 715 | 670 | 680 | 587 | 650 |
| Streptococcus thermophilus CBT ST3 | 310 | 712 | 660 | 620 | 600 | 668 |
| Pediococcus pentosaceus CBT SL4 | 346 | 732 | 680 | 655 | 670 | 680 |

(unit: ×10⁷ cfu/ml)

### Experimental Example 3: Experimental for Selection of Nitrogen Source

As shown in Table 5 below, 2 parts by weight of each nitrogen source was added to 100 parts by weight of BL broth, thereby preparing media.

TABLE 5

| | Control | Yeast | Nonfat dry | Isolated soy |
|---|---|---|---|---|
| Strain name | (BL broth) | extract | milk | protein |
| Lactobacillus casei CBT LC5 | 0% | 2% | 2% | 2% |
| Lactobacillus plantarum CBT LP3 | 0% | 2% | 2% | 2% |
| Lactobacillus rhamnosus CBT LR5 | 0% | 2% | 2% | 2% |
| Bifidobacterium longum CBT BG7 | 0% | 2% | 2% | 2% |
| Bifidobacterium lactis CBT BL3 | 0% | 2% | 2% | 2% |
| Bifidobacterium bifidum CBT BF3 | 0% | 2% | 2% | 2% |
| Streptococcus thermophilus CBT ST3 | 0% | 2% | 2% | 2% |
| Pediococcus pentosaceus CBT SL4 | 0% | 2% | 2% | 2% |

0.1 to 1 part by weight of each starter culture was inoculated into 100 parts by weight of each prepared medium, and then cultured for 20 hours while the pH was maintained at about 5.0 to 6.5. Next, the viable cell count of each culture was measured, and the results of the measurement are summarized in Table 6 below. As a result, it was confirmed that culture with yeast extract or isolated soy protein exhibited a better effect on the growth of the lactic acid bacteria than the other nitrogen sources. Thus, yeast extract or isolated soy protein was selected for a subsequent experiment (Table 6).

TABLE 6

| | Control | Yeast | Nonfat dry | Isolated soy |
|---|---|---|---|---|
| Strain name | (BL broth) | extract | milk | protein |
| Lactobacillus casei CBT LC5 | 355 | 670 | 560 | 680 |
| Lactobacillus plantarum CBT LP3 | 325 | 820 | 615 | 885 |
| Lactobacillus rhamnosus CBT LR5 | 226 | 750 | 645 | 890 |
| Bifidobacterium longum CBT BG7 | 342 | 752 | 670 | 725 |
| Bifidobacterium lactis CBT BL3 | 442 | 815 | 775 | 806 |
| Bifidobacterium bifidum CBT BF3 | 301 | 670 | 604 | 770 |
| Streptococcus thermophilus CBT ST3 | 295 | 620 | 805 | 720 |
| Pediococcus pentosaceus CBT SL4 | 315 | 722 | 715 | 780 |

(unit: ×10⁷ cfu/ml)

### Experimental Example 4: Experiment for Combination of Nitrogen Sources

As shown in Table 7 below, yeast extract and isolated soy protein were mixed together at the indicated ratios, and then 2 parts by weight of each mixture was added to 100 parts by weight of BL broth, thereby preparing media.

TABLE 7

| | Control | 1:1 | 1:2 | 1:3 | 2:1 | 3:1 |
|---|---|---|---|---|---|---|
| Strain name | (BL broth) | 50%:50% | 33%:67% | 25%:75% | 67%:33% | 75%:25% |
| Lactobacillus casei CBT LC5 | 0% | 2% | 2% | 2% | 2% | 2% |
| Lactobacillus plantarum CBT LP3 | 0% | 2% | 2% | 2% | 2% | 2% |
| Lactobacillus rhamnosus CBT LR5 | 0% | 2% | 2% | 2% | 2% | 2% |
| Bifidobacterium longum CBT BG7 | 0% | 2% | 2% | 2% | 2% | 2% |
| Bifidobacterium lactis CBT BL3 | 0% | 2% | 2% | 2% | 2% | 2% |
| Bifidobacterium bifidum CBT BF3 | 0% | 2% | 2% | 2% | 2% | 2% |
| Streptococcus thermophilus CBT ST3 | 0% | 2% | 2% | 2% | 2% | 2% |
| Pediococcus pentosaceus CBT SL4 | 0% | 2% | 2% | 2% | 2% | 2% |

0.1 to 1 part by weight of each starter culture was inoculated into 100 parts by weight of each prepared medium, and then cultured for 20 hours while the pH was maintained at about 5.0 to 6.5. Next, the viable cell count of each culture was measured, and the results of the measurement are summarized in Table 8 below. As a result, it was confirmed that culture with yeast extract and isolated soy protein at a mixing ratio of 3:1 (75%: 25%) exhibited a better effect on the growth of the lactic acid bacteria than culture at the other mixing ratios. Thus, the mixing ratio of 3:1 was selected as a mixing ratio for a subsequent experiment (Table 8).

TABLE 8

| | Control | 1:1 | 1:2 | 1:3 | 2:1 | 3:1 |
|---|---|---|---|---|---|---|
| Strain name | (BL broth) | 50%:50% | 33%:67% | 25%:75% | 67%:33% | 75%:25% |
| Lactobacillus casei CBT LC5 | 348 | 690 | 670 | 680 | 690 | 702 |
| Lactobacillus plantarum CBT LP3 | 318 | 830 | 863 | 872 | 886 | 895 |
| Lactobacillus rhamnosus CBT LR5 | 235 | 760 | 845 | 854 | 863 | 906 |
| Bifidobacterium longum CBT BG7 | 318 | 762 | 733 | 745 | 755 | 825 |
| Bifidobacterium lactis CBT BL3 | 422 | 822 | 785 | 796 | 803 | 816 |
| Bifidobacterium bifidum CBT BF3 | 317 | 702 | 725 | 734 | 752 | 802 |
| Streptococcus thermophilus CBT ST3 | 322 | 705 | 715 | 720 | 725 | 810 |
| Pediococcus pentosaceus CBT SL4 | 336 | 780 | 765 | 740 | 750 | 833 |

(unit: ×10⁷ cfu/ml)

### Experimental Example 5: Experiment for Selection of Optimal Mixture Composition Comprising Four Different Components

As shown in Table 9 below, glucose, fructooligosaccharide, yeast extract and isolated soy protein were mixed together at the indicated ratios, and then 2 parts by weight of each of the mixtures was added to 100 parts by weight of BL broth, thereby preparing media.

TABLE 9

| | Control | | | | | |
|---|---|---|---|---|---|---|
| | (BL | | | | | |
| Strain name | broth) | 25%:25%:25%:25% | 20%:20%:30%:30% | 30%:30%:20%:20% | 40%:40%:10%:10% | 40%:40%:15%:5% |
| Lactobacillus casei CBT | 0% | 2% | 2% | 2% | 2% | 2% |
| LC5 | | | | | | |
| Lactobacillus plantarum CBT | 0% | 2% | 2% | 2% | 2% | 2% |
| LP3 | | | | | | |
| Lactobacillus rhamnosus CBT | 0% | 2% | 2% | 2% | 2% | 2% |
| LR5 | | | | | | |
| Bifidobacterium longum CBT | 0% | 2% | 2% | 2% | 2% | 2% |
| BG7 | | | | | | |
| Bifidobacterium lactis CBT | 0% | 2% | 2% | 2% | 2% | 2% |
| BL3 | | | | | | |
| Bifidobacterium bifidum CBT | 0% | 2% | 2% | 2% | 2% | 2% |
| BF3 | | | | | | |
| Streptococcus thermophilus CBT | 0% | 2% | 2% | 2% | 2% | 2% |
| ST3 | | | | | | |
| Pediococcus pentosaceus CBT | 0% | 2% | 2% | 2% | 2% | 2% |
| SL4 | | | | | | |

0.1 to 1 part by weight of each starter culture was inoculated into 100 parts by weight of each of the prepared media, and then cultured for 20 hours while the pH was maintained at about 5.0 to 6.5. Next, the viable cell count of each culture was measured, and the results of the measurement are summarized in Table 10 below. As a result, it was confirmed that, as the content of the carbon source was higher than the content of the nitrogen source, the effect of the carbon source and the nitrogen source on the growth of the lactic acid bacteria increased, and when 40 wt % glucose, 40 wt % fructooligosaccharide, 15 wt % yeast extract and 5 wt % isolated soy protein were mixed together, the mixture exhibited the best effect on the growth of the lactic acid bacteria.

TABLE 10

| | Control | | | | | |
|---|---|---|---|---|---|---|
| | (BL | | | | | |
| Strain name | broth) | 25%:25%:25%:25% | 20%:20%:30%:30% | 30%:30%:20%:20% | 40%:40%:10%:10% | 40%:40%:15%:5% |
| Lactobacillus casei CBT | 344 | 770 | 760 | 820 | 970 | 1,070 |
| LC5 | | | | | | |
| Lactobacillus plantarum CBT | 334 | 746 | 735 | 810 | 935 | 1,040 |
| LP3 | | | | | | |
| Lactobacillus rhamnosus CBT | 243 | 762 | 732 | 832 | 975 | 1,030 |
| LR5 | | | | | | |
| Bifidobacterium longum CBT | 312 | 740 | 725 | 825 | 925 | 1,050 |
| BG7 | | | | | | |
| Bifidobacterium lactis CBT | 419 | 730 | 720 | 760 | 910 | 1,100 |
| BL3 | | | | | | |
| Bifidobacterium bifidum CBT | 305 | 700 | 710 | 840 | 922 | 1,020 |
| BF3 | | | | | | |
| Streptococcus thermophilus CBT | 345 | 680 | 700 | 752 | 912 | 1,120 |
| ST3 | | | | | | |
| Pediococcus pentosaceus CBT | 332 | 775 | 732 | 796 | 948 | 1,050 |
| SL4 | | | | | | |

(unit: ×10⁷ cfu/ml)

### Experimental Example 6: Experiment for Verification of Optimal Mixture Composition

To finally verify the effect of the optimal mixture composition, mixtures of two or three of the four components were compared with the final mixture composition. As shown in Table 11 below, glucose, fructooligosaccharide, yeast extract and isolated soy protein were mixed together at various ratios as follows: Comparative Example 1 (not comprising), Comparative Example 2 (75% yeast extract and 25% isolated soy protein), Comparative Example 3 (80% glucose, 15% yeast extract, and 5% isolated soy protein), Comparative Example 4 (80% fructooligosaccharide, 15% yeast extract, and 5% isolated soy protein), and Example (40% glucose, 40% fructooligosaccharide, 15% yeast extract, and 5% isolated soy protein). Then, 2 parts by weight of each of the mixtures was added to 100 parts by weight of BL broth, thereby preparing media.

TABLE 11

| | Comparative | | | | |
|---|---|---|---|---|---|
| | Example 1 | Comparative | Comparative | Comparative | |
| Strain name | (not comprising) | Example 2 | Example 3 | Example 4 | Example |
| Lactobacillus casei CBT LC5 | 0% | 2% | 2% | 2% | 2% |
| Lactobacillus plantarum CBT LP3 | 0% | 2% | 2% | 2% | 2% |
| Lactobacillus rhamnosus CBT LR5 | 0% | 2% | 2% | 2% | 2% |
| Bifidobacterium longum CBT BG7 | 0% | 2% | 2% | 2% | 2% |
| Bifidobacterium lactis CBT BL3 | 0% | 2% | 2% | 2% | 2% |
| Bifidobacterium bifidum CBT BF3 | 0% | 2% | 2% | 2% | 2% |
| Streptococcus thermophilus CBT ST3 | 0% | 2% | 2% | 2% | 2% |
| Pediococcus pentosaceus CBT SL4 | 0% | 2% | 2% | 2% | 2% |

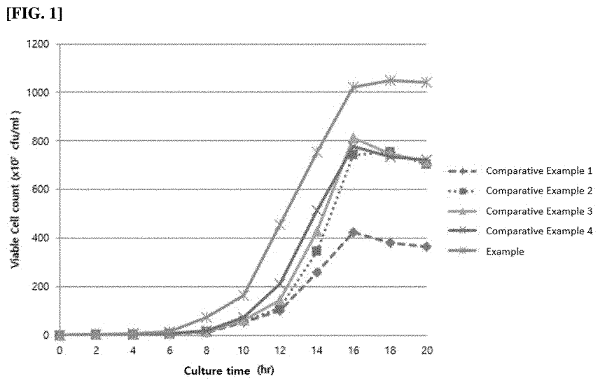

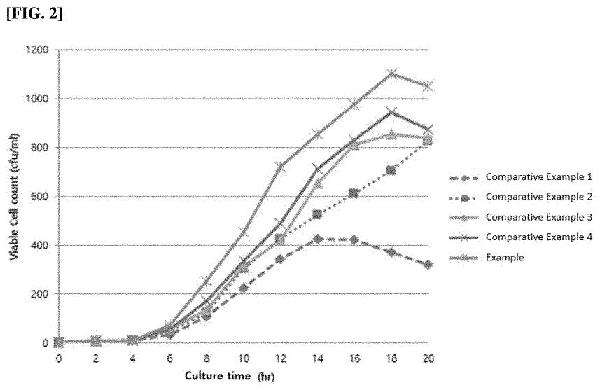

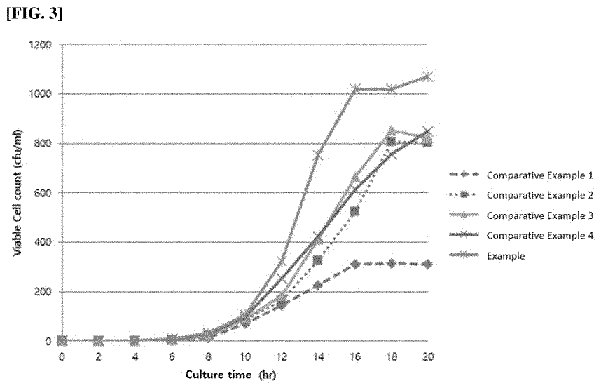

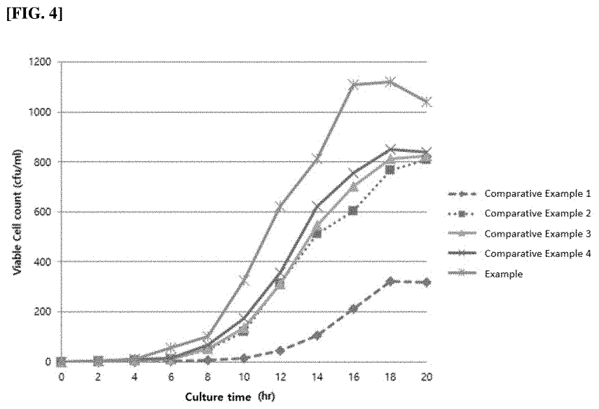

0.1 to 1 part by weight of each starter culture was inoculated into 100 parts by weight of each of the prepared media, and then cultured for 20 hours while the pH was maintained at about 5.0 to 6.5. Next, the viable cell count of each culture was measured, and the results of the measurement are summarized in Table 12 below. In addition, the growth curve of each strain was analyzed by measuring the number of viable cells at 2-hour intervals for 20 hours (FIGS. 1 to 4). As a result, it was confirmed that culture with the composition of the Example (the composition comprising the mixture of glucose, fructooligosaccharide, yeast extract and isolated soy protein) exhibited an excellent effect of promoting the growth of all the eight types of lactic acid bacteria compared to culture with each of the compositions of Comparative Examples 1 to 4, and it could be confirmed that the composition of the Example exhibited not only the effects of activating the growth of the lactic acid bacteria and promoting cell division, but also the stability effect of allowing the proliferated lactic acid bacteria to survive for a long time (Table 12 and FIGS. 1 to 4).

TABLE 12

| | Comparative | | | | |
|---|---|---|---|---|---|
| | Example 1 | Comparative | Comparative | Comparative | |
| Strain name | (not comprising) | Example 2 | Example 3 | Example 4 | Example |
| Lactobacillus casei CBT LC5 | 363 | 702 | 715 | 722 | 1,040 |
| Lactobacillus plantarum CBT LP3 | 327 | 895 | 920 | 915 | 1,030 |
| Lactobacillus rhamnosus CBT LR5 | 252 | 906 | 935 | 944 | 1,050 |
| Bifidobacterium longum CBT BG7 | 316 | 825 | 837 | 872 | 1,050 |
| Bifidobacterium lactis CBT BL3 | 399 | 816 | 845 | 882 | 1,090 |
| Bifidobacterium bifidum CBT BF3 | 312 | 802 | 822 | 850 | 1,070 |
| Streptococcus thermophilus CBT ST3 | 317 | 810 | 825 | 838 | 1,040 |
| Pediococcus pentosaceus CBT SL4 | 343 | 833 | 826 | 872 | 1,000 |

(unit: ×10⁷ cfu/ml)

As described in detail above, the composition for promoting the growth of lactic acid bacteria comprising growth factors according to the present disclosure may promote the growth of lactic acid bacteria, and thus may be used universally as a composition for promoting the growth of various lactic acid bacteria.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

[Accession Number]

Depository authority: Korea Research Institute of Bioscience and Biotechnology

Accession number: KCTC12199BP

Deposit date: Apr. 27, 2012

Depository authority: Korea Research Institute of Bioscience and Biotechnology

Accession number: KCTC12200BP

Deposit date: Apr. 27, 2012

Depository authority: Korea Research Institute of Bioscience and Biotechnology

Accession number: KCTC11904BP

Deposit date: Mar. 30, 2011

Depository authority: Korea Research Institute of Bioscience and Biotechnology

Accession number: KCTC12398BP

Deposit date: Apr. 5, 2013

Depository authority: Korea Research Institute of Bioscience and Biotechnology

Accession number: KCTC10782BP

Deposit date: Mar. 16, 2005

Depository authority: Korea Research Institute of Bioscience and Biotechnology

Accession number: KCTC12202BP

Deposit date: Apr. 27, 2012

Depository authority: Korea Research Institute of Bioscience and Biotechnology

Accession number: KCTC10297BP

Deposit date: Jun. 25, 2002

Depository authority: Korea Research Institute of Bioscience and Biotechnology

Accession number: KCTC11870BP

Deposit date: Feb. 17, 2011

What is claimed is:

1. A composition for promoting the growth of lactic acid bacteria, the composition comprising:
a carbon source comprising glucose and fructooligosaccharide; and
a nitrogen source comprising yeast extract and isolated soy protein.

2. The composition of claim 1, wherein the composition comprise 20 to 40 wt % of the glucose, 20 to 40 wt % of the fructooligosaccharide, 10 to 30 wt % of the yeast extract, and 5 to 30 wt % of the isolated soy protein.

3. The composition of claim 1, wherein the carbon source comprises the glucose and the fructooligosaccharide at a weight ratio of 1:3 to 3:1.

4. The composition of claim 1, wherein the nitrogen source comprises the yeast extract and the isolated soy protein at a weight ratio of 1:3 to 3:1.

5. The composition of claim 1, wherein the lactic acid bacteria are one or more lactic acid bacteria selected from the group consisting of lactic acid bacteria of the genus Lactobacillus, lactic acid bacteria of the genus Bifidobacterium, lactic acid bacteria of the genus Streptococcus, and lactic acid bacteria of the genus Pediococcus.

6. The composition of claim 5, wherein the lactic acid bacteria of the genus Lactobacillus are one or more lactic acid bacteria selected from the group consisting of Lactobacillus casei, Lactobacillus plantarum and Lactobacillus rhamnosus.

7. The composition of claim 5, wherein the lactic acid bacteria of the genus Bifidobacterium are one or more lactic acid bacteria selected from the group consisting of Bifidobacterium longum, Bifidobacterium lactis and Bifidobacterium bifidum.

8. The composition of claim 5, wherein the lactic acid bacteria of the genus Streptococcus are Streptococcus thermophilus.

9. The composition of claim 5, wherein the lactic acid bacteria of the genus Pediococcus are Pediococcus pentosaceus.
